SEQUENCE LISTING (202 sequences)

SEQ ID NO: 1 - RNA, 20 nt, synthetic construct
  source 1..20: mol_type = other RNA
tgatccagaacccgggcccc

SEQ ID NO: 2 - RNA, 20 nt, synthetic construct
  source 1..20: mol_type = other RNA
gagcgtgcgcgaagcgatcc

SEQ ID NO: 3 - RNA, 20 nt, synthetic construct
  source 1..20: mol_type = other RNA
cgatccagaacccgggcccc

SEQ ID NO: 4 - RNA, 20 nt, synthetic construct
  source 1..20: mol_type = other RNA
ggcggcggcggcggcgaggc

SEQ ID NO: 5 - RNA, 20 nt, synthetic construct
  source 1..20: mol_type = other RNA
ggcggcggcggcggcgaggc

SEQ ID NO: 6 - RNA, 20 nt, synthetic construct
  source 1..20: mol_type = other RNA
ggcggcggcggcggcgaggc

SEQ ID NO: 7 - RNA, 20 nt, synthetic construct
  source 1..20: mol_type = other RNA
ggcggcggcggcggcgaggc

SEQ ID NO: 8 - RNA, 20 nt, synthetic construct
  source 1..20: mol_type = other RNA
ggcggcggcggcggcgaggc

SEQ ID NO: 9 - RNA, 20 nt, synthetic construct
  source 1..20: mol_type = other RNA
ggcggcggcggcggcgaggc

SEQ ID NO: 10 - RNA, 20 nt, synthetic construct
  source 1..20: mol_type = other RNA
cggcggcggcggcggcgagg

SEQ ID NO: 11 - RNA, 20 nt, synthetic construct
  source 1..20: mol_type = other RNA
cggcggcggcggcggcgagg

SEQ ID NO: 12 - RNA, 20 nt, synthetic construct
  source 1..20: mol_type = other RNA
cggcggcggcggcggcgagg

SEQ ID NO: 13 - RNA, 20 nt, synthetic construct
  source 1..20: mol_type = other RNA
cggcggcggcggcggcgagg

SEQ ID NO: 14 - RNA, 20 nt, synthetic construct
  source 1..20: mol_type = other RNA
cggcggcggcggcggcgagg

SEQ ID NO: 15 - RNA, 20 nt, synthetic construct
  source 1..20: mol_type = other RNA
cggcggcggcggcggcgagg

SEQ ID NO: 16 - RNA, 20 nt, synthetic construct
  source 1..20: mol_type = other RNA
aagtgggccaaggccttgcc

SEQ ID NO: 17 - RNA, 20 nt, synthetic construct
  source 1..20: mol_type = other RNA
aagtgggccaaggccttgcc

SEQ ID NO: 18 - RNA, 20 nt, synthetic construct
  source 1..20: mol_type = other RNA
aagtgggccaaggccttgcc

SEQ ID NO: 19 - RNA, 20 nt, synthetic construct
  source 1..20: mol_type = other RNA
tctccagcttgatgcgagcg

SEQ ID NO: 20 - RNA, 20 nt, synthetic construct
  source 1..20: mol_type = other RNA
tctccagcttgatgcgagcg

SEQ ID NO: 21 - RNA, 20 nt, synthetic construct
  source 1..20: mol_type = other RNA
tctccagcttgatgcgagcg

SEQ ID NO: 22 - RNA, 20 nt, synthetic construct
  source 1..20: mol_type = other RNA
tctccagcttgatgcgagcg

SEQ ID NO: 23 - RNA, 20 nt, synthetic construct
  source 1..20: mol_type = other RNA
tctccagcttgatgcgagcg

SEQ ID NO: 24 - RNA, 20 nt, synthetic construct
  source 1..20: mol_type = other RNA
tctccagcttgatgcgagcg

SEQ ID NO: 25 - RNA, 20 nt, synthetic construct
  source 1..20: mol_type = other RNA
acacactacacctggctcaa

SEQ ID NO: 26 - RNA, 20 nt, synthetic construct
  source 1..20: mol_type = other RNA
acacactacacctggctcaa

SEQ ID NO: 27 - RNA, 20 nt, synthetic construct
  source 1..20: mol_type = other RNA
acacactacacctggctcaa

SEQ ID NO: 28 - RNA, 20 nt, synthetic construct
  source 1..20: mol_type = other RNA
tagccccctacggctacact

SEQ ID NO: 29 - RNA, 20 nt, synthetic construct
  source 1..20: mol_type = other RNA
tagccccctacggctacact

SEQ ID NO: 30 - RNA, 20 nt, synthetic construct
  source 1..20: mol_type = other RNA
tagccccctacggctacact

SEQ ID NO: 31 - RNA, 20 nt, synthetic construct
  source 1..20: mol_type = other RNA
tagccccctacggctacact

SEQ ID NO: 32 - RNA, 20 nt, synthetic construct
  source 1..20: mol_type = other RNA
tagccccctacggctacact

SEQ ID NO: 33 - RNA, 20 nt, synthetic construct
  source 1..20: mol_type = other RNA
tagccccctacggctacact

SEQ ID NO: 34 - RNA, 20 nt, synthetic construct
  source 1..20: mol_type = other RNA
gctcgcggcctctgggtgcc

SEQ ID NO: 35 - RNA, 20 nt, synthetic construct
  source 1..20: mol_type = other RNA
gctcgcggcctctgggtgcc

SEQ ID NO: 36 - RNA, 20 nt, synthetic construct
  source 1..20: mol_type = other RNA
gctcgcggcctctgggtgcc

SEQ ID NO: 37 - RNA, 20 nt, synthetic construct
  source 1..20: mol_type = other RNA
gctcgcggcctctgggtgcc

SEQ ID NO: 38 - RNA, 20 nt, synthetic construct
  source 1..20: mol_type = other RNA
gctcgcggcctctgggtgcc

SEQ ID NO: 39 - RNA, 20 nt, synthetic construct
  source 1..20: mol_type = other RNA
cagcagcgggagagcgaggg

SEQ ID NO: 40 - RNA, 20 nt, synthetic construct
  source 1..20: mol_type = other RNA
cagcagcgggagagcgaggg

SEQ ID NO: 41 - RNA, 20 nt, synthetic construct
  source 1..20: mol_type = other RNA
cagcagcgggagagcgaggg

SEQ ID NO: 42 - RNA, 20 nt, synthetic construct
  source 1..20: mol_type = other RNA
cagcagcgggagagcgaggg

SEQ ID NO: 43 - RNA, 20 nt, synthetic construct
  source 1..20: mol_type = other RNA
cagcagcgggagagcgaggg

SEQ ID NO: 44 - RNA, 20 nt, synthetic construct
  source 1..20: mol_type = other RNA
cagcagcgggagagcgaggg

SEQ ID NO: 45 - RNA, 20 nt, synthetic construct
  source 1..20: mol_type = other RNA
tctgggacgcaacctctctc

SEQ ID NO: 46 - RNA, 20 nt, synthetic construct
  source 1..20: mol_type = other RNA
tctgggacgcaacctctctc

SEQ ID NO: 47 - RNA, 20 nt, synthetic construct
  source 1..20: mol_type = other RNA
tctgggacgcaacctctctc

SEQ ID NO: 48 - RNA, 20 nt, synthetic construct
  source 1..20: mol_type = other RNA
tctgggacgcaacctctctc

SEQ ID NO: 49 - RNA, 20 nt, synthetic construct
  source 1..20: mol_type = other RNA
tctgggacgcaacctctctc

SEQ ID NO: 50 - RNA, 20 nt, synthetic construct
  source 1..20: mol_type = other RNA
ctgctgcggcagccccttgc

SEQ ID NO: 51 - RNA, 20 nt, synthetic construct
  source 1..20: mol_type = other RNA
ctgctgcggcagccccttgc

SEQ ID NO: 52 - RNA, 20 nt, synthetic construct
  source 1..20: mol_type = other RNA
ctgctgcggcagccccttgc

SEQ ID NO: 53 - RNA, 20 nt, synthetic construct
  source 1..20: mol_type = other RNA
ctgctgcggcagccccttgc

SEQ ID NO: 54 - RNA, 20 nt, synthetic construct
  source 1..20: mol_type = other RNA
ctgctgcggcagccccttgc

SEQ ID NO: 55 - RNA, 20 nt, synthetic construct
  source 1..20: mol_type = other RNA
gccatccaaactcttgagag

SEQ ID NO: 56 - RNA, 20 nt, synthetic construct
  source 1..20: mol_type = other RNA
gccatccaaactcttgagag

SEQ ID NO: 57 - RNA, 20 nt, synthetic construct
  source 1..20: mol_type = other RNA
gtcctggaagccattgagcc

SEQ ID NO: 58 - RNA, 20 nt, synthetic construct
  source 1..20: mol_type = other RNA
gtcctggaagccattgagcc

SEQ ID NO: 59 - RNA, 20 nt, synthetic construct
  source 1..20: mol_type = other RNA
gtcctggaagccattgagcc

SEQ ID NO: 60 - RNA, 20 nt, synthetic construct
  source 1..20: mol_type = other RNA
ctgctgcagcagcagcaaac

SEQ ID NO: 61 - RNA, 20 nt, synthetic construct
  source 1..20: mol_type = other RNA
ctgctgcagcagcagcaaac

SEQ ID NO: 62 - RNA, 20 nt, synthetic construct
  source 1..20: mol_type = other RNA
ctgctgcagcagcagcaaac

SEQ ID NO: 63 - RNA, 20 nt, synthetic construct
  source 1..20: mol_type = other RNA
ctgctgcagcagcagcaaac

SEQ ID NO: 64 - RNA, 20 nt, synthetic construct
  source 1..20: mol_type = other RNA
ctgctgcagcagcagcaaac

SEQ ID NO: 65 - RNA, 20 nt, synthetic construct
  source 1..20: mol_type = other RNA
cggccccctcaggggctggc

SEQ ID NO: 66 - RNA, 20 nt, synthetic construct
  source 1..20: mol_type = other RNA
cggccccctcaggggctggc

SEQ ID NO: 67 - RNA, 20 nt, synthetic construct
  source 1..20: mol_type = other RNA
cggccccctcaggggctggc

SEQ ID NO: 68 - RNA, 20 nt, synthetic construct
  source 1..20: mol_type = other RNA
cggccccctcaggggctggc

SEQ ID NO: 69 - RNA, 20 nt, synthetic construct
  source 1..20: mol_type = other RNA
cggccccctcaggggctggc

SEQ ID NO: 70 - RNA, 20 nt, synthetic construct
  source 1..20: mol_type = other RNA
cggccccctcaggggctggc

SEQ ID NO: 71 - RNA, 20 nt, synthetic construct
  source 1..20: mol_type = other RNA
ctggagtgccaccccgagag

SEQ ID NO: 72 - RNA, 20 nt, synthetic construct
  source 1..20: mol_type = other RNA
ctggagtgccaccccgagag

SEQ ID NO: 73 - RNA, 20 nt, synthetic construct
  source 1..20: mol_type = other RNA
ctggagtgccaccccgagag

SEQ ID NO: 74 - RNA, 20 nt, synthetic construct
  source 1..20: mol_type = other RNA
ctggagtgccaccccgagag

SEQ ID NO: 75 - RNA, 20 nt, synthetic construct
  source 1..20: mol_type = other RNA
ctggagtgccaccccgagag

SEQ ID NO: 76 - RNA, 20 nt, synthetic construct
  source 1..20: mol_type = other RNA
tccggagtagctatccatcc

SEQ ID NO: 77 - RNA, 20 nt, synthetic construct
  source 1..20: mol_type = other RNA
tccggagtagctatccatcc

SEQ ID NO: 78 - RNA, 20 nt, synthetic construct
  source 1..20: mol_type = other RNA
tccggagtagctatccatcc

SEQ ID NO: 79 - RNA, 20 nt, synthetic construct
  source 1..20: mol_type = other RNA
tccggagtagctatccatcc

SEQ ID NO: 80 - RNA, 20 nt, synthetic construct
  source 1..20: mol_type = other RNA
tccggagtagctatccatcc

SEQ ID NO: 81 - RNA, 20 nt, synthetic construct
  source 1..20: mol_type = other RNA
tccggagtagctatccatcc

SEQ ID NO: 82 - RNA, 20 nt, synthetic construct
  source 1..20: mol_type = other RNA
gcagcagcagcgggagagcg

SEQ ID NO: 83 - RNA, 20 nt, synthetic construct
  source 1..20: mol_type = other RNA
gcagcagcagcgggagagcg

SEQ ID NO: 84 - RNA, 20 nt, synthetic construct
  source 1..20: mol_type = other RNA
gcagcagcagcgggagagcg

SEQ ID NO: 85 - RNA, 20 nt, synthetic construct
  source 1..20: mol_type = other RNA
gcagcagcagcgggagagcg

SEQ ID NO: 86 - RNA, 20 nt, synthetic construct
  source 1..20: mol_type = other RNA
gcagcagcagcgggagagcg

SEQ ID NO: 87 - RNA, 20 nt, synthetic construct
  source 1..20: mol_type = other RNA
gcagcagcagcgggagagcg

SEQ ID NO: 88 - RNA, 20 nt, synthetic construct
  source 1..20: mol_type = other RNA
cggcggagggggcggcggtc

SEQ ID NO: 89 - RNA, 20 nt, synthetic construct
  source 1..20: mol_type = other RNA
cggcggagggggcggcggtc

SEQ ID NO: 90 - RNA, 20 nt, synthetic construct
  source 1..20: mol_type = other RNA
cggcggagggggcggcggtc

SEQ ID NO: 91 - RNA, 20 nt, synthetic construct
  source 1..20: mol_type = other RNA
cggcggagggggcggcggtc

SEQ ID NO: 92 - RNA, 20 nt, synthetic construct
  source 1..20: mol_type = other RNA
cggcggagggggcggcggtc

SEQ ID NO: 93 - RNA, 20 nt, synthetic construct
  source 1..20: mol_type = other RNA
cggcggagggggcggcggtc

SEQ ID NO: 94 - RNA, 20 nt, synthetic construct
  source 1..20: mol_type = other RNA
tctcccgctgctgctgcctt

SEQ ID NO: 95 - RNA, 20 nt, synthetic construct
  source 1..20: mol_type = other RNA
tctcccgctgctgctgcctt

SEQ ID NO: 96 - RNA, 20 nt, synthetic construct
  source 1..20: mol_type = other RNA
tctcccgctgctgctgcctt

SEQ ID NO: 97 - RNA, 20 nt, synthetic construct
  source 1..20: mol_type = other RNA
tctcccgctgctgctgcctt

SEQ ID NO: 98 - RNA, 20 nt, synthetic construct
  source 1..20: mol_type = other RNA
tctcccgctgctgctgcctt

SEQ ID NO: 99 - RNA, 20 nt, synthetic construct
  source 1..20: mol_type = other RNA
tctcccgctgctgctgcctt

SEQ ID NO: 100 - RNA, 20 nt, synthetic construct
  source 1..20: mol_type = other RNA
ggggacctggcgagcctgca

SEQ ID NO: 101 - RNA, 20 nt, synthetic construct
  source 1..20: mol_type = other RNA
ggggacctggcgagcctgca

SEQ ID NO: 102 - RNA, 20 nt, synthetic construct
  source 1..20: mol_type = other RNA
ggggacctggcgagcctgca

SEQ ID NO: 103 - RNA, 20 nt, synthetic construct
  source 1..20: mol_type = other RNA
ggggacctggcgagcctgca

SEQ ID NO: 104 - RNA, 20 nt, synthetic construct
  source 1..20: mol_type = other RNA
ggggacctggcgagcctgca

SEQ ID NO: 105 - RNA, 20 nt, synthetic construct
  source 1..20: mol_type = other RNA
ggggacctggcgagcctgca

SEQ ID NO: 106 - RNA, 20 nt, synthetic construct
  source 1..20: mol_type = other RNA
cccgggccccaggcacccag

SEQ ID NO: 107 - RNA, 20 nt, synthetic construct
  source 1..20: mol_type = other RNA
cccgggccccaggcacccag

SEQ ID NO: 108 - RNA, 20 nt, synthetic construct
  source 1..20: mol_type = other RNA
cccgggccccaggcacccag

SEQ ID NO: 109 - RNA, 20 nt, synthetic construct
  source 1..20: mol_type = other RNA
cccgggccccaggcacccag

SEQ ID NO: 110 - RNA, 20 nt, synthetic construct
  source 1..20: mol_type = other RNA
cccgggccccaggcacccag

SEQ ID NO: 111 - RNA, 20 nt, synthetic construct
  source 1..20: mol_type = other RNA
ttccaggacattcagaaaga

SEQ ID NO: 112 - RNA, 20 nt, synthetic construct
  source 1..20: mol_type = other RNA
ttccaggacattcagaaaga

SEQ ID NO: 113 - RNA, 20 nt, synthetic construct
  source 1..20: mol_type = other RNA
ttccaggacattcagaaaga

SEQ ID NO: 114 - RNA, 20 nt, synthetic construct
  source 1..20: mol_type = other RNA
ctctgggacgcaacctctct

SEQ ID NO: 115 - RNA, 20 nt, synthetic construct
  source 1..20: mol_type = other RNA
ctctgggacgcaacctctct

SEQ ID NO: 116 - RNA, 20 nt, synthetic construct
  source 1..20: mol_type = other RNA
ctctgggacgcaacctctct

SEQ ID NO: 117 - RNA, 20 nt, synthetic construct
  source 1..20: mol_type = other RNA
ctctgggacgcaacctctct

SEQ ID NO: 118 - RNA, 20 nt, synthetic construct
  source 1..20: mol_type = other RNA
ctctgggacgcaacctctct

SEQ ID NO: 119 - RNA, 20 nt, synthetic construct
  source 1..20: mol_type = other RNA
ctccggacttgtagagagac

SEQ ID NO: 120 - RNA, 20 nt, synthetic construct
  source 1..20: mol_type = other RNA
ctccggacttgtagagagac

SEQ ID NO: 121 - RNA, 20 nt, synthetic construct
  source 1..20: mol_type = other RNA
ctccggacttgtagagagac

SEQ ID NO: 122 - RNA, 20 nt, synthetic construct
  source 1..20: mol_type = other RNA
ctccggacttgtagagagac

SEQ ID NO: 123 - RNA, 20 nt, synthetic construct
  source 1..20: mol_type = other RNA
ctccggacttgtagagagac

SEQ ID NO: 124 - RNA, 20 nt, synthetic construct
  source 1..20: mol_type = other RNA
ctccggacttgtagagagac

SEQ ID NO: 125 - RNA, 20 nt, synthetic construct
  source 1..20: mol_type = other RNA
ccgccgtggccgccagcaag

SEQ ID NO: 126 - RNA, 20 nt, synthetic construct
  source 1..20: mol_type = other RNA
ccgccgtggccgccagcaag

SEQ ID NO: 127 - RNA, 20 nt, synthetic construct
  source 1..20: mol_type = other RNA
ccgccgtggccgccagcaag

SEQ ID NO: 128 - RNA, 20 nt, synthetic construct
  source 1..20: mol_type = other RNA
ccgccgtggccgccagcaag

SEQ ID NO: 129 - RNA, 20 nt, synthetic construct
  source 1..20: mol_type = other RNA
ccgccgtggccgccagcaag

SEQ ID NO: 130 - RNA, 20 nt, synthetic construct
  source 1..20: mol_type = other RNA
gcttgtacacgtggtcaagt

SEQ ID NO: 131 - RNA, 20 nt, synthetic construct
  source 1..20: mol_type = other RNA
gcttgtacacgtggtcaagt

SEQ ID NO: 132 - RNA, 20 nt, synthetic construct
  source 1..20: mol_type = other RNA
gcttgtacacgtggtcaagt

SEQ ID NO: 133 - RNA, 20 nt, synthetic construct
  source 1..20: mol_type = other RNA
tagaggccccacaggctacc

SEQ ID NO: 134 - RNA, 20 nt, synthetic construct
  source 1..20: mol_type = other RNA
tagaggccccacaggctacc

SEQ ID NO: 135 - RNA, 20 nt, synthetic construct
  source 1..20: mol_type = other RNA
tagaggccccacaggctacc

SEQ ID NO: 136 - RNA, 20 nt, synthetic construct
  source 1..20: mol_type = other RNA
tagaggccccacaggctacc

SEQ ID NO: 137 - RNA, 20 nt, synthetic construct
  source 1..20: mol_type = other RNA
tagaggccccacaggctacc

SEQ ID NO: 138 - RNA, 20 nt, synthetic construct
  source 1..20: mol_type = other RNA
gatgcttgcaattgccaacc

SEQ ID NO: 139 - RNA, 20 nt, synthetic construct
  source 1..20: mol_type = other RNA
ggactacggcagcgcctggg

SEQ ID NO: 140 - RNA, 20 nt, synthetic construct
  source 1..20: mol_type = other RNA
ggactacggcagcgcctggg

SEQ ID NO: 141 - RNA, 20 nt, synthetic construct
  source 1..20: mol_type = other RNA
ggactacggcagcgcctggg

SEQ ID NO: 142 - RNA, 20 nt, synthetic construct
  source 1..20: mol_type = other RNA
ggactacggcagcgcctggg

SEQ ID NO: 143 - RNA, 20 nt, synthetic construct
  source 1..20: mol_type = other RNA
ggactacggcagcgcctggg

SEQ ID NO: 144 - RNA, 20 nt, synthetic construct
  source 1..20: mol_type = other RNA
ggactacggcagcgcctggg

SEQ ID NO: 145 - RNA, 20 nt, synthetic construct
  source 1..20: mol_type = other RNA
tccaaggacaattacttagg

SEQ ID NO: 146 - RNA, 20 nt, synthetic construct
  source 1..20: mol_type = other RNA
tccaaggacaattacttagg

SEQ ID NO: 147 - RNA, 20 nt, synthetic construct
  source 1..20: mol_type = other RNA
tccaaggacaattacttagg

SEQ ID NO: 148 - RNA, 20 nt, synthetic construct
  source 1..20: mol_type = other RNA
tccaaggacaattacttagg

SEQ ID NO: 149 - RNA, 20 nt, synthetic construct
  source 1..20: mol_type = other RNA
tccaaggacaattacttagg

SEQ ID NO: 150 - RNA, 20 nt, synthetic construct
  source 1..20: mol_type = other RNA
tccaaggacaattacttagg

SEQ ID NO: 151 - RNA, 20 nt, synthetic construct
  source 1..20: mol_type = other RNA
agcttgtacacgtggtcaag

SEQ ID NO: 152 - RNA, 20 nt, synthetic construct
  source 1..20: mol_type = other RNA
agcttgtacacgtggtcaag

SEQ ID NO: 153 - RNA, 20 nt, synthetic construct
  source 1..20: mol_type = other RNA
agcttgtacacgtggtcaag

SEQ ID NO: 154 - RNA, 20 nt, synthetic construct
  source 1..20: mol_type = other RNA
taaccccaagccatactgca

SEQ ID NO: 155 - RNA, 20 nt, synthetic construct
  source 1..20: mol_type = other RNA
ttgacttctagcaaataaat

SEQ ID NO: 156 - RNA, 20 nt, synthetic construct
  source 1..20: mol_type = other RNA
ggtgaaggatcgccagccca

SEQ ID NO: 157 - RNA, 20 nt, synthetic construct
  source 1..20: mol_type = other RNA
ggtgaaggatcgccagccca

SEQ ID NO: 158 - RNA, 20 nt, synthetic construct
  source 1..20: mol_type = other RNA
ccgctgcacccgcgccatgc

SEQ ID NO: 159 - RNA, 20 nt, synthetic construct
  source 1..20: mol_type = other RNA
ccgctgcacccgcgccatgc

SEQ ID NO: 160 - RNA, 20 nt, synthetic construct
  source 1..20: mol_type = other RNA
ccgctgcacccgcgccatgc

SEQ ID NO: 161 - RNA, 20 nt, synthetic construct
  source 1..20: mol_type = other RNA
ccgctgcacccgcgccatgc

SEQ ID NO: 162 - RNA, 20 nt, synthetic construct
  source 1..20: mol_type = other RNA
ccgctgcacccgcgccatgc

SEQ ID NO: 163 - RNA, 20 nt, synthetic construct
  source 1..20: mol_type = other RNA
ccgctgcacccgcgccatgc

SEQ ID NO: 164 - RNA, 20 nt, synthetic construct
  source 1..20: mol_type = other RNA
gaagcagggatgactctggg

SEQ ID NO: 165 - RNA, 20 nt, synthetic construct
  source 1..20: mol_type = other RNA
gaagcagggatgactctggg

SEQ ID NO: 166 - RNA, 20 nt, synthetic construct
  source 1..20: mol_type = other RNA
gaagcagggatgactctggg

SEQ ID NO: 167 - RNA, 20 nt, synthetic construct
  source 1..20: mol_type = other RNA
gaagcagggatgactctggg

SEQ ID NO: 168 - RNA, 20 nt, synthetic construct
  source 1..20: mol_type = other RNA
cgctggactacggcagcgcc

SEQ ID NO: 169 - RNA, 20 nt, synthetic construct
  source 1..20: mol_type = other RNA
cgctggactacggcagcgcc

SEQ ID NO: 170 - RNA, 20 nt, synthetic construct
  source 1..20: mol_type = other RNA
cgctggactacggcagcgcc

SEQ ID NO: 171 - RNA, 20 nt, synthetic construct
  source 1..20: mol_type = other RNA
cgctggactacggcagcgcc

SEQ ID NO: 172 - RNA, 20 nt, synthetic construct
  source 1..20: mol_type = other RNA
cgctggactacggcagcgcc

SEQ ID NO: 173 - RNA, 20 nt, synthetic construct
  source 1..20: mol_type = other RNA
cgctggactacggcagcgcc

SEQ ID NO: 174 - RNA, 20 nt, synthetic construct
  source 1..20: mol_type = other RNA
ctcattgaaaaccagatcag

SEQ ID NO: 175 - RNA, 20 nt, synthetic construct
  source 1..20: mol_type = other RNA
ctcattgaaaaccagatcag

SEQ ID NO: 176 - RNA, 20 nt, synthetic construct
  source 1..20: mol_type = other RNA
tccccacgctcgcatcaagc

SEQ ID NO: 177 - RNA, 20 nt, synthetic construct
  source 1..20: mol_type = other RNA
tccccacgctcgcatcaagc

SEQ ID NO: 178 - RNA, 20 nt, synthetic construct
  source 1..20: mol_type = other RNA
tccccacgctcgcatcaagc

SEQ ID NO: 179 - RNA, 20 nt, synthetic construct
  source 1..20: mol_type = other RNA
tccccacgctcgcatcaagc

SEQ ID NO: 180 - RNA, 20 nt, synthetic construct
  source 1..20: mol_type = other RNA
tccccacgctcgcatcaagc

SEQ ID NO: 181 - RNA, 20 nt, synthetic construct
  source 1..20: mol_type = other RNA
tccccacgctcgcatcaagc

SEQ ID NO: 182 - RNA, 20 nt, synthetic construct
  source 1..20: mol_type = other RNA
acaggtacttctgtttccct

SEQ ID NO: 183 - RNA, 20 nt, synthetic construct
  source 1..20: mol_type = other RNA
acaggtacttctgtttccct

SEQ ID NO: 184 - RNA, 20 nt, synthetic construct
  source 1..20: mol_type = other RNA
acaggtacttctgtttccct

SEQ ID NO: 185 - RNA, 20 nt, synthetic construct
  source 1..20: mol_type = other RNA
acaggtacttctgtttccct

SEQ ID NO: 186 - RNA, 20 nt, synthetic construct
  source 1..20: mol_type = other RNA
acaggtacttctgtttccct

SEQ ID NO: 187 - RNA, 20 nt, synthetic construct
  source 1..20: mol_type = other RNA
tccaggacattcagaaagat

SEQ ID NO: 188 - RNA, 20 nt, synthetic construct
  source 1..20: mol_type = other RNA
tccaggacattcagaaagat

SEQ ID NO: 189 - RNA, 20 nt, synthetic construct
  source 1..20: mol_type = other RNA
tccaggacattcagaaagat

SEQ ID NO: 190 - RNA, 20 nt, synthetic construct
  source 1..20: mol_type = other RNA
ggctgtgaagagagtgtgcc

SEQ ID NO: 191 - RNA, 20 nt, synthetic construct
  source 1..20: mol_type = other RNA
ggctgtgaagagagtgtgcc

SEQ ID NO: 192 - RNA, 20 nt, synthetic construct
  source 1..20: mol_type = other RNA
ggctgtgaagagagtgtgcc

SEQ ID NO: 193 - RNA, 20 nt, synthetic construct
  source 1..20: mol_type = other RNA
ggctgtgaagagagtgtgcc

SEQ ID NO: 194 - RNA, 20 nt, synthetic construct
  source 1..20: mol_type = other RNA
ggctgtgaagagagtgtgcc

SEQ ID NO: 195 - RNA, 20 nt, synthetic construct
  source 1..20: mol_type = other RNA
ggctgtgaagagagtgtgcc

SEQ ID NO: 196 - RNA, 20 nt, synthetic construct
  source 1..20: mol_type = other RNA
agccgccgtggccgccagca

SEQ ID NO: 197 - RNA, 20 nt, synthetic construct
  source 1..20: mol_type = other RNA
agccgccgtggccgccagca

SEQ ID NO: 198 - RNA, 20 nt, synthetic construct
  source 1..20: mol_type = other RNA
agccgccgtggccgccagca

SEQ ID NO: 199 - RNA, 20 nt, synthetic construct
  source 1..20: mol_type = other RNA
agccgccgtggccgccagca

SEQ ID NO: 200 - RNA, 20 nt, synthetic construct
  source 1..20: mol_type = other RNA
agccgccgtggccgccagca

SEQ ID NO: 201 - RNA, 20 nt, synthetic construct
  source 1..20: mol_type = other RNA
tgtcattcagtactcctgga

SEQ ID NO: 202 - RNA, 20 nt, synthetic construct
  source 1..20: mol_type = other RNA
tgtcattcagtactcctgga

(19) United States
(12) Patent Application Publication
(10) Pub. No.: US 2024/0173435 A1
(43) Pub. Date: May 30, 2024

(54) GENE DELIVERY FOR PREVENTION AND TREATMENT OF BALDNESS

(71) Applicant: NEW JERSEY INSTITUTE OF TECHNOLOGY, Newark, NJ (US)

(72) Inventors: Xiaoyang Xu, Livingston, NJ (US); Zhongyu Li, Harrison, NJ (US); William Ho, Newark, NJ (US)

(73) Assignee: NEW JERSEY INSTITUTE OF TECHNOLOGY, Newark, NJ (US)

(21) Appl. No.: 18/524,574

(22) Filed: Nov. 30, 2023

Related U.S. Application Data
(60) Provisional application No. 63/428,769, filed on Nov. 30, 2022.

(51) Int. Cl. A61K 48/00; A61P 17/14; C12N 9/22; C12N 15/63

(52) U.S. Cl. CPC A61K 48/0058; A61P 17/14; C12N 9/22; C12N 15/63

(57) ABSTRACT

Ionizable cationic lipid compounds have an amine moiety from amino alcohols and a lipid moiety from a lipid synthesized via esterification. The ionizable cationic lipid compounds which comprise an amino alcohol mediated ionizable cationic lipid compound are useful for in vivo or in vitro delivery of one or more nucleic acid agents including DNA, siRNA, a microRNA, an mRNA, a RNAi, and a plasmid.

### CROSS-REFERENCE TO RELATED APPLICATION

The present application claims the benefit of priority, under 35 U.S.C. 119 (e) to U.S. Provisional Appl. No. 63/428,769, filed Nov. 30, 2022, which is hereby incorporated by reference in its entirety.

### STATEMENT REGARDING FEDERALLY SPONSORED RESEARCH

This invention was made with government support under Agreement No. 2001606 awarded by National Science Foundation. The government has certain rights in the invention.

### STATEMENT REGARDING ELECTRONIC FILING OF A SEQUENCE LISTING

A Sequence Listing is submitted under 37 C.F.R. § 1.831. The Sequence Listing is in XML format, generated on Nov. 22, 2023, entitled NJI0130-00US Sequence Listing (ST.26).xml, 175K bytes in size. The Sequence Listing material, as identified above, is incorporated by reference into the specification.

### TECHNICAL FIELD

The present disclosure relates to gene therapy. In particular, the present disclosure relates to the synthesis, development, and use of nucleic acid constructs and nano-encapsulation of the same for gene delivery, prevention, and treatment of baldness.

### BACKGROUND

Hair loss is a pressing concern for the majority of men and a significant number of women as they age. Balding or androgenic alopecia is an extremely common hair loss disorder affecting roughly 30% of Caucasian men by 30 years of age, 50% by 50 years, and 80% by 70 years, with similar trends yet lower percentages in other races (Birch, Messenger et al. 2001, Lolli, Pallotti et al. 2017, Fabbrocini, Cantelli et al. 2018). The disorder is so prevalent that it is unlikely that anyone has not known a person or who themselves is not balding, or already bald. The defining characteristic of androgenic alopecia is the progressive thinning of hair due to androgenic and genetic factors, leading to hair loss in a defined pattern (Rathnayake and Sinclair 2010, York, Meah et al. 2020). Sufferers experience lowered self-esteem, distress, and other psychological and physiological effects, which have a primarily negative impact on their personal, social, and romantic lives. In most societies, balding is seen as an indicator of low vigor, low vitality, and aging (Messenger, 2008).

Hair loss proceeds when the male hormone testosterone is converted into dihydrotestosterone (DHT) by an enzyme called 5-alpha reductase, and the DHT binds to cells and their androgen receptors in the scalp, which react by progressively thinning hair through follicle and hair miniaturization. Over months and years, eventually the hairs fall out or become so thin and small to be un-noticeable.

Although early treatments were not effective, modern science has brought the field forward a significant amount, though not yet having reached a cure. The modern treatments for baldness are divided into two main categories: chemical treatments and hair transplants. Chemical treatments include minoxidil, which is a vasodilator, (York, Meah et al. 2020) and finasteride, which is a DHT enzyme inhibitor. Chemical treatments may not work for all people. They require twice daily treatment for the rest of one's lifetime, which can be a significant burden on the patient, and present significant issues with regard to the patient's compliance. More specifically, finasteride is a global 5αR2 inhibitor, which has a concerning risk of causing adverse and unwanted sexual effects, including gynecomastia, reduced libido, erectile dysfunction, and ejaculatory disorders.

Hair transplants require painstaking transplantation of follicles one at a time. Such transplants have a low success rate and carry an expensive price tag. Up to 90% of all transplanted follicles will die, and since the underlying problem is not treated, the hair around the area, which is composed of non-transplanted hair, will continue to thin, leading to the necessity of further painful and expensive treatments. Additionally, the operations take place in multiple sessions due to healing time, taking up to two years for full completion of a single treatment regimen. Often, many men, frustrated by the slow and constant treatment, fatalistically accept hair loss and stop treatment altogether. There is a need for a one-time treatment, which permanently solves hair loss and promotes regrowth. The one-time treatment would serve as the “holy grail” of baldness or androgenic alopecia.

### SUMMARY

Disclosed is a new paradigm of baldness treatment. The present disclosure provides a CRISPR-Cas nanoparticle delivery system that comprises a designed short guide RNA (sgRNA) that targets the androgen receptor gene. In one or more embodiments, the sgRNA of the present disclosure, instead of the conventional solution approach of blocking testosterone hormone, targets the androgen receptors that bind to DHT. In one or more embodiments, the CRISPR-Cas lipid nanoparticle (LNP) delivery system of the present disclosure locally delivers an anti-androgenic alopecia treatment, which allows for rescue from hair loss. In one or more embodiments, the treatment method according to the present disclosure is a one-time administration of the treatment as compared to daily application of other known baldness treatments. The CRISPR-Cas nanoparticle delivery system according to one or more embodiments of the present invention is capable of stopping or inhibiting the androgen receptor at the source (e.g., scalp) instead of blocking testosterone/DHT in the entire body, which leads to side effects. Additionally, the CRISPR-Cas nanoparticle delivery system according to one or more embodiments of the present disclosure provides a non-painful topical administration without surgery. The CRISPR/Cas nanoparticle delivery system according to one or more embodiments the present disclosure, by taking advantage of new mRNA and CRISPR-Cas technologies, can potentially solve many problems associated with the known chemical and surgical treatments available today for baldness in a simple and ingenious manner, offering one of the first successful uses of CRISPR technology for the treatment of any disease worldwide.

A gene editing method known as CRISPR-Cas9 is an innovation in genetic engineering. CRISPR-Cas9 may modify, delete, or correct specific areas of DNA. CRISPR-Cas9 generally includes Cas9 nuclease, an enzyme that cuts DNA, and a guide RNA (gRNA) whose sequence directs Cas9 nuclease to a specific location in the DNA where the edit should be made. One or more embodiments of the present invention provides a nanoparticle delivery system that encapsulates a novel CRISPR-Cas/sgRNA system that induces mutation in the androgen receptor gene.

A first aspect of the invention pertains to a nucleic acid construct comprising a domain hybridizing with early coding regions of the androgen receptor gene.

A second aspect of the invention pertains to a polynucleotide encoding a nucleic acid construct comprising a domain hybridizing with early coding regions of the androgen receptor gene and a CRISPR-Cas nuclease, wherein the nucleic acid construct and the CRISPR-Cas nuclease form a complex that hybridizes with the androgen receptor gene and induces mutation in the gene.

A third aspect of the invention pertains to a composition comprising a) a nanoparticle delivery system and b) at least one of a nucleic acid construct comprising a domain hybridizing with early coding regions of the androgen receptor gene, an expression cassette or vector comprising the same, a polynucleotide encoding a nucleic acid construct comprising a domain hybridizing with early coding regions of the androgen receptor gene and a CRISPR-Cas nuclease, and an expression cassette or vector comprising the same.

A fourth aspect of the invention pertains to a method of treating baldness or hair loss in a subject in need thereof, the method comprising administering to the subject a therapeutically effective amount of a nucleic acid construct comprising a domain hybridizing with early coding regions of the androgen receptor gene and/or an expression cassette or vector comprising the same, and a CRSIPR/Cas nuclease and/or an expression cassette or vector comprising the same, wherein the nucleic acid construct forms a complex with the CRISPR/Cas nuclease, the complex hybridizing with early coding regions of the androgen receptor gene, wherein the complex knocks out the gene.

A fifth aspect of the invention pertains to a kit comprising the compositions described herein, optionally with instructions for use thereof.

The aspects of the present invention are described with more details below.

### SEQUENCES

SEQ ID NOs: 1-2 exemplify sgRNA nucleotide sequences of the present disclosure that are used for CRISPR/Cas9 treatment of baldness in mammals.

SEQ ID NOs: 3-202 exemplify designed sgRNA nucleotide sequences of the present disclosure that have potentially high efficacy of hybridizing with the androgen receptor gene.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Further features of the present invention will become apparent from the following written description and the accompanying figures, in which:

FIG. 1. Graphical depiction showing the size and distribution of lipid nanoparticles (LNPs) containing sgRNA/Cas mRNA, in accordance with one or more embodiments of the present disclosure;

FIG. 2. (Top left) Sample image of transfection of HEK 293T cells at 48 h with plasmids containing GFP reporter gene as well as sgRNA and CRISPR Cas9. (Bottom left) Sample FACS cell sorting data indicating percentage of GFP positive cells, which were utilized for sequencing analysis. (Top right) Sample DNA sequence trace of sgRNAs. Vertical dashed line represents the predicted CRISPR edit site. (Bottom right) Knockout percentages of sgRNAs, calculated by ICE analysis (n=5). This indicates percentage of transfected cells that show a knockout mutation (insertion or deletion);

FIG. 3. Mouse skin treated with fluorescent DSPC-FITC labeled LNPs. LNPs were administered topically on the skin of mice after depilation and images captured 24 h after treatment, indicating penetration depth of LNPs are 100-150 microns. Hair follicles in mice clearly accumulate fluorescent LNPs. Scale bar 100 microns;

FIG. 4. LNPs can transdermally deliver mRNA, and expression is detected. Delivery is presumably through the pores generated by depilation cream in C57BL/6 mice, which dissolves hair follicles that previously occupied the area. LNPs loaded with luciferase mRNA (left), empty (no mRNA) LNPS (middle) and nude mice (no hair removal);

FIG. 5. LNP/CRISPR dose dependence study. C57/BL mice (age 6-8 weeks) were depilated on the back and treated with LNPs containing sgRNA and CRISPR-Cas9 mRNA at 1:1 molar ratio, one time. On dl until the end of the experiment, all mice were treated with DHT (10E-8 M) daily (200 uL, topical) to inhibit the hair formation. From left to right, 5 ug sgRNA, 10 ug sgRNA, 20 ug sgRNA, no LNP, and empty LNP;

FIG. 6. Immunostain of androgen receptor expression in mice 3 d (left) and 6 d (right) after treatment after hair removal. Control untreated mice (top row) and 20 ug sgRNA targeting androgen receptor (bottom row) are shown;

FIG. 7. Histology of mouse skin and hair after 12 d, highlighting the hair follicle density and number. Control untreated (left column) and 20 ug sgRNA NP treated (right column) are shown;

FIG. 8a shows a chemical synthesis route of AA3-DLin through CALB enzyme-assisted esterification according to one or more embodiments; and

FIG. 8b illustrates schematic views of an ionizable cationic lipid nanoparticle (LNP) and a therapeutic lipid nanoparticle, in which a nucleic acid agent (e.g., sgRNA) is encapsulated, according to one or more embodiments.

### DETAILED DESCRIPTION

Different embodiments of the present disclosure are described herein with reference to examples and drawings. It is noted that the present disclosure is not a listing of all the possible ways the claimed invention can be used. The limitations according to embodiments of the present disclosure may be incorporated in other embodiments or may not be present in other embodiments. Therefore, any combination and/or permutation of the embodiments is envisioned. Other objects and features will become apparent from the following detailed description considered in conjunction with the accompanying drawings. It is to be understood that the drawings are designed as an illustration only and not as a definition of the limits of the present disclosure.

Unless specifically stated, the embodiments of the present disclosure can be used in any combination with another. For example, an embodiment that comprises limitations X, Y, Z may be X, Y, Z, or a combination thereof.

The term “and/or” is to be interpreted to include any and all the possible combinations of one or more of the limitations. The term “or” is to be interpreted as alternative, meaning lack of combination thereof.

The term “about” is to be interpreted as a tolerance of ±0.1%, ±1%, ±5%, ±10%, or ±20% of a specified value.

The terms “increase”, “enhance”, “improve”, “extend”, and “grow” describe an increase of at least about 10%, 20%, 50%, 75%, 100%, 200%, 300%, 400%, 500% or any value therebetween when compared to a control value.

The terms “decrease”, “diminish”, “reduce”, and “shrink”, describe a decrease of at least about 10%, 20%, 50%, 75%, 100%, 200%, 300%, 400%, 500% or any value therebetween when compared to a control value.

The phrase “between X and Y” is to be interpreted as including measurable values of X, Y and any value therebetween.

The term “heterologous” is referred to as nucleotide sequence that does not naturally occur in a host cell into which it is administered.

The terms “nucleic acid”, “nucleic sequence”, “nucleic acid construct” and “polynucleotide” may be used interchangeably and refer to a polymer of nucleotides from the 5′ to 3′ end and may include an RNA or DNA.

Nucleic sequences of the present disclosure are provided in 5′ to 3′ direction (from left to right).

The term “mutation” refers to point mutation, frameshift mutation, base edition, and/or truncation.

The term “gene” refers to a polynucleotide that can serve as a template to produce, for example, mRNA, anti-sense RNA, etc. A gene may encompass coding and non-coding regions (e.g., promoters, termination sequences, introns, exons, protein-coding regions, etc.). A gene may refer to non-isolated and isolated forms, the former meaning that the gene resides in its natural environment inside a cell, the latter meaning free from cellular material and/or reagents used for isolation synthesis, and cell growth.

The term “complementary” means the degree of binding of two polynucleotides through base pairing. For example, base “T” pairs with “A” and “G” pairs with “C”. Base pairing between two strands of polynucleotides can be complete (100% complementary) or substantial (less than 100% complementary). Complementary as used herein mean 100% and less (e.g., 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, or less)

A “portion” refers to a nucleotide sequence that lacks nucleotides when compared to a reference sequence. For example, the length of a portion may be reduced by 1, 2, 3, 4, 5, 6, 7, 8, 9, 10, 11, 12, 13, 14, 15, or more nucleotides. A portion of a polynucleotide may comprise, consist, or essentially consist of 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, or less of a reference polynucleotide.

The term “homologue” may refer to polynucleotides/proteins with substantial similarity/identity in sequence and function with a reference polynucleotide/protein. Homologous polynucleotides/proteins of the present disclosure have substantially similar/identical sequences, for example at least 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75% identical. According to some embodiments of the present disclosure, substantial similarity/identity is present over a nucleotide/polypeptide sequence with about 10 to about 15 nucleotides/amino acids, about 10 to about 20 nucleotides/amino acids, about 10 to about 25 nucleotides/amino acids, about 10 to about 30 nucleotides/amino acids, about 10 to about 40 nucleotides/amino acids, about 10 to about 50 nucleotides/amino acids, about 10 to about 60 nucleotides/amino acids, about 10 to about 70 nucleotides/amino acids, about 10 to about 80 nucleotides/amino acids, about 10 to about 90 nucleotides/amino acids, about 10 to about 100 nucleotides/amino acids or more.

The term “complex”, according to some embodiments of the present disclosure may refer to a polynucleotide (e.g., sgRNA), a portion, or a homologue thereof, functionally linked to a protein (e.g. CRISPR-Cas nuclease) or a homologue thereof “Functionally linked” means to affect a meaningful biological function (e.g., expression, transcription, upregulation, downregulation, mutation, and the like). In some embodiments, nucleotide sequences can also be functionally linked to each other on a single nucleotide acid construct.

In some embodiments, the CRISPR-Cas nucleases that can form a complex with sgRNA of the present disclosure can have mutation in their catalytic active site and therefore may have decreased activity or no activity when compared to a CRISPR-Cas without a mutation.

The term “promoter” refers to a nucleotide sequence that regulates the transcription of a nucleotide sequence that is functionally linked to the promoter. A polynucleotide regulated by a promoter may encode a polynucleotide, a protein or and/or a polynucleotide/protein complex. Various types of promoter are known to one having ordinary skill in the art. For example, tissue specific promoters can be used for transdermal expression of polynucleotides, proteins, and/or polynucleotide/protein complexes according to some embodiments of the present disclosure. According to some embodiments, the nucleic acid construct of the present disclosure may be functionally linked to a promoter. The nucleic acids of the present disclosure may be functionally linked to translational and/or transcriptional promoters.

The nucleic acid constructs, polynucleotides, expression cassettes and/or vectors according to some embodiments of the present disclosure are codon optimized for expression in mammalian cells. More specifically, according to some embodiments, nucleic constructs, polynucleotides, expression cassettes and/or vectors are optimized for expression in mice and human cells. According to some embodiments, the codon optimized, nucleic acid constructs, polynucleotides, expression cassettes and/or vectors are, for example 100%, 99.9%, 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, or less identical to nucleic constructs, polynucleotides, expression cassettes and/or vectors that are not codon-optimized.

The term “repeat sequence” may refer to any repeat sequence of a CRISPR-Cas locus (e.g., Class I, and Class II) or an identical repeat sequence thereof which is functionally linked with a suitable CRISPR-Cas nuclease or the nuclease encoded by the polypeptide according to embodiments of the present disclosure. The term “repeat sequence” may refer to a wild-type or synthesized sequence, or a portion thereof that is functionally linked to a suitable CRISPR-Cas nuclease (e.g., Class I, Class II) or the nuclease encoded by the polypeptide according to embodiments of the present disclosure. The CRISPR-Cas nucleases that can be used with sgRNA of the present disclosure are not limited to Class I and Class II and any suitable nuclease can be used.

The synthesized “sgRNA” according to some embodiments of the present disclosure forms a complex with CRIPSR-Cas9 nuclease. The sgRNA/Cas9 complex acts as molecular “scissors” that cut DNA at the site the designed single guide RNAs (sgRNAs) targets, and subsequent DNA repair processes will introduce errors, which in effect, cause a knock-out of the androgen receptor gene, if the errors are placed in the right coding areas.

The term “spacer sequence” may refer to a nucleic acid sequence that hybridizes to a target nucleic acid. Spacer sequences according to embodiments of the present disclosure can be at least 75% complementary (e.g., 100%, 99.9%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, or less) to a target (e.g., the androgen gene). In some embodiments, the spacer may be completely (e.g., 100%) or substantially complementary to a portion of the target nucleic acid.

A “guide nucleic acid”, “guide RNA”, “sgRNA” or “gRNA” may refer to a nucleic acid that comprises at least one spacer sequence that is complementary to a target nucleic acid. A sgRNA may further comprise at least a repeat sequence or a portion thereof. In some embodiments the repeat sequence may be linked to the 5′ end of the spacer sequence. In some embodiments, the repeat sequence may be linked to 3′ end of the spacer sequence. In some embodiments, the repeat sequence may be linked to both 5′ and 3′ end of the spacer sequence.

A “protospacer adjacent motif”, or “PAM” may refer to a nucleic acid sequence on a target DNA adjacent to a protospacer sequence that is recognized by a CRISPR-Cas nuclease. A PAM is recognized by a nuclease and allows the nuclease to modify the target DNA. According to some embodiments, a PAM may be located upstream (5′ end) and/or downstream (3′ end) of a protospacer. Although a list of PAMs for each sgRNA according to the present disclosure is provided, additional PAMs can be used with sgRNAs. Such PAMs may be designed through experimental or computational approaches.

A “protospacer” may refer to a target nucleotide sequence or a portion thereof wherein a sgRNA is completely or substantially hybridized to.

The singulars “a”, “an”, and “the” may refer to plural forms unless indicated otherwise.

According to some embodiments, a vector and/or an expression cassette comprises the nucleic acid construct (e.g., sgRNA) of the present disclosure. According to some embodiments, a vector and/or an expression cassette comprises the polynucleotide of the present disclosure. The expression cassette and/or vector according to some embodiments of the present disclosure may comprise heterologous components. For example, a promoter from the same or different host functionally linked to the polypeptide and/or polynucleotide according to some embodiments of the present disclosure. In some embodiments, the nucleic acid construct (e.g., sgRNA) may be provided on a different expression cassette and/or vector than a nuclease (e.g., Cas nuclease).

In some embodiments, the present disclosure provides a cell comprising at least one of the nucleic acid constructs of the present disclosure, an expression cassette or vector comprising the same, a polynucleotide encoding the nucleic acid construct of the present disclosure and a CRISPR-Cas nuclease, and an expression cassette or vector comprising the same. The nucleic construct, polynucleotide, expression cassette/vector of the present disclosure can be introduced to a host cell by any known method.

A lipid nanoparticle delivery system according to some embodiments of the present invention comprises a payload of the nucleic acid construct, polynucleotide, and/or expression cassette/vector of the present disclosure.

In some embodiments, the nanoparticle delivery system comprises a lipid nanoparticle (LNP) comprising an ionizable cationic lipid compound comprising a reaction product of an amino alcohol and one or more lipid acids having from 4 to 26 carbons (C4-C26). The lipid nanoparticle (LNP) further comprises one or more other lipid components selected from the group consisting of helper phospholipids, PEGylated lipids, cholesterol, and a combination thereof. In some embodiments, the amino alcohol comprises one or more hydroxyl (OH) groups. In some embodiments the one or more lipid acids is selected from the group consisting of octanoic acid (C8), decanoic acid (C10), dodecanoic acid (C12), tetradecanoic acid (C14), hexadecanoic acid (C16), octadecanoic acid (C18), oleic acid (C18:1), linoleic acid (C18:2), and a combination thereof. In one particular embodiment, the amino alcohol comprises a piperazine derivative, and the one or more lipid acids is selected from the group consisting of octanoic acid (C8), decanoic acid (C10), dodecanoic acid (C12), tetradecanoic acid (C14), hexadecanoic acid (C16), octadecanoic acid (C18), oleic acid (C18:1), linoleic acid (C18:2), and a combination thereof. In a more particular embodiment, the amino alcohol comprises 1,4-Bis(2-hydroxyethyl) piperazine, the lipid acid is linoleic acid, and the one or more other lipid components is selected from the group consisting of 1,2-dioleoyl-sn-glycero-3-phosphoethanolamine (DOPE), cholesterol, 1,2-dimyristoyl-rac-glycero-3-methoxypolyethylene glycol (DMG-PEG), and a combination thereof. In one embodiment, the helper phospholipid is selected from the group consisting of 1,2-dioleoyl-sn-glycero-3-phosphoethanolamine (DOPE), 1,2-distearoyl-sn-glycero-3-phosphocholine (DSPC), dioleoylphosphatidylcholine (DOPC), and a combination thereof. In one embodiment, the PEGylated lipid is 1,2-dimyristoyl-rac-glycero-3-methoxypolyethylene glycol (DMG-PEG).

### EXAMPLES

### Example 1

### Design, Synthesis, and Screening of an Ionizable Cationic Lipid Library

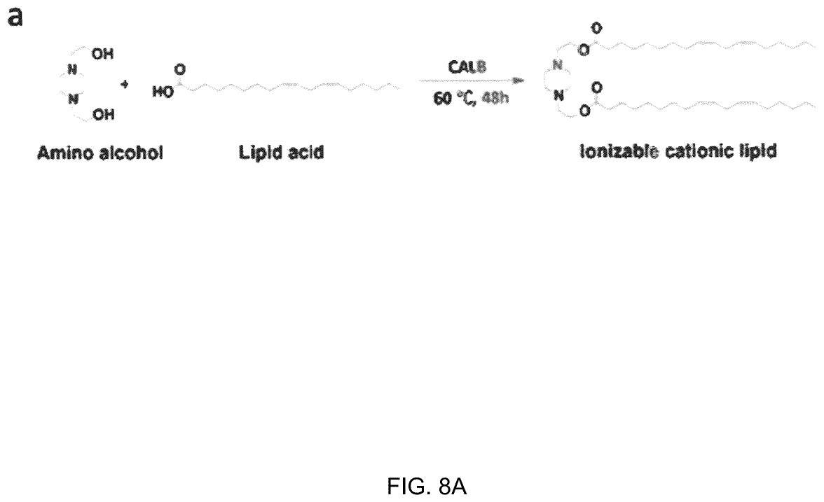

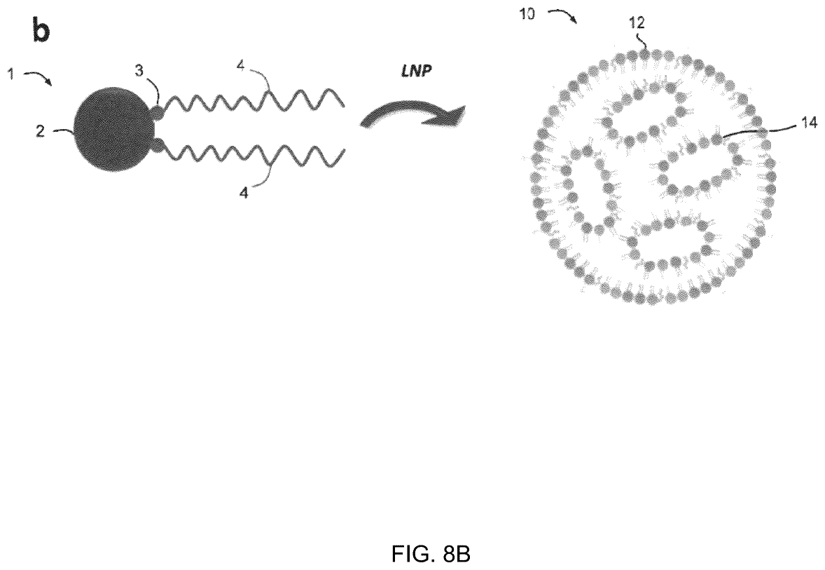

An ionizable cationic lipid is generally composed of three parts: (1) hydrophilic headgroups containing one or multiple ionizable amines for condensing negatively charged mRNA; (2) hydrophobic hydrocarbon chains capable of promoting self-assembly and phospholipid membrane fusion; (3) degradable ester linkers connecting the headgroups with hydrocarbon chains to potentially lower systemic cytotoxicity (FIG. 8b). A new library of ionizable cationic lipids is designed, where the ionizable amine headgroups originated from different amino alcohols and the hydrocarbon lipid chains were derived from commercially available lipid acids. Through CALB enzyme-assisted reaction, the hydroxyl groups were reacted with carboxylic acids via one-step high-efficiency esterification (FIG. 8a) and an 18*8 library of lipid-like materials was synthesized by varying amino alcohols and lipid acids (ACS Nano, 16 (11) 18936-18950). Preliminarily, these lipid-like materials with DOPE, cholesterol, and DMG-PEG were fabricated at a molar ratio of 50:10:38.5:1.5, which was the most widely used formulation to form LNPs and delivered luciferase encoded mRNA (mLuc) in vitro to generate a luciferase expression heat map of lipid-like materials.

The top-performing ionizable cationic lipid was screened and termed as AA3-DLin, which is chemically composed of 1,4-Bis(2-hydroxyethyl) piperazine amine headgroups connected with two linoleic lipids by ester linkers as shown in FIG. 8. The AA3-DLin lipids were successfully synthesized by high-efficiency CALB-mediated catalytic esterification and characterized by electrospray ionization (ESI) mass spectrometry, which showed a strong, clear and single peak denoting AA3-DLin with molecular weight (MW) of 699 and up to 96% purity. Furthermore, nuclear magnetic resonance (NMR) spectroscopy and Fourier-transform infrared spectroscopy (FTIR) revealed the chemical structure of AA3-DLin accordingly. In one or more embodiments, the ionizable cationic lipid molecules comprise a molecular weight in a range of from about 200 to about 2000 Daltons, including all values and subranges therebetween.

FIG. 8a shows a chemical synthesis route of AA3-DLin through CALB enzyme-assisted esterification, according to one or more embodiments, to prepare amino alcohol mediated ionizable cationic lipid compounds. The reaction of amino alcohol and lipid acid is conducted at 60° C. for 48 hours in the presence of an enzyme (e.g., CALB). FIG. 8b illustrates a schematic view of an ionizable cationic lipid 1 comprising an amino alcohol mediated ionizable cationic lipid compound, according to one or more embodiments. An amine head 2 supplied by the amino alcohol is attached by an ester linker 3 to lipid chains 4. FIG. 8b also illustrates a schematic view of a therapeutic lipid nanoparticle 10. The therapeutic lipid nanoparticle 10 comprises an LNP fabricated by using one or more amino alcohol mediated ionizable cationic lipid compounds, with other lipid components, which assemble to form an outer shell 12 encapsulating a primary core, which comprises a nucleic acid-based agent 16 such as sgRNA. In one embodiment, the outer shell 12 comprises an assembly of an amino alcohol mediated ionizable cationic lipid compound, with a phospholipid, a cholesterol, and a PEGylated lipid.

### Example 2

Fabrication of mRNA Loaded Lipid Nanoparticles (LNPs) by Microfluidic Chip Device

In one embodiment, a microfluidic chip device was applied to fabricate the LNPs. The microfluidic chip device used for AA3-DLin LNP fabrication was reported in the following study: Li, Z., Zhang, X. Q., Ho, W., Li, F., Gao, M., Bai, X., & Xu, X. (2022). Enzyme-Catalyzed One-Step Synthesis of Ionizable Cationic Lipids for Lipid Nanoparticle-Based mRNA COVID-19 Vaccines. ACS nano.) The ethanol phase contained a mixture of AA3-DLin, DOPE, cholesterol, and DMG-PEG 2000 at a molar ratio of 40:40:25:0.5. The aqueous phase was prepared in 25 mM NaOAc buffer (pH 5.0). The ethanol and aqueous phases were loaded in two different syringes at a volume ratio of 1:3 and a fixed mRNA/AA3-DLin weight ratio of 1:20 for in vitro and 1:10 for in vivo, respectively. The two phases were mixed in a microfluidic chip device using syringe pumps with pre-set pump rates. The resulting LNPs were subsequently incubated for 30 min at room temperature before dialysis against 1×PBS in a Pur-A-Lyzer Midi Dialysis Kit (MWCO 3.5 kDa) for 2 h at 4° C. to remove ethanol.

### Example 3

Cas9, sgRNA Selection and Design

Cas9 mRNA was purchased from Tri-Link Biotechnologies. Guide RNAs, which target the 1st coding exon in both human and mouse AR genes, were designed so that any frameshift mutation would result in early termination of translation. All gRNAs were obtained from Synthego with “end-modified” synthesis where the first and last three bases of these gRNA were synthesized using 2′-O-Methyl-nucleosides and joined via 3′ phosphorothioate bonds.

TABLE 1
Designed sgRNAs for CRISPR/Cas9
treatment of baldness used in study.

| | | | gRNA |
|---|---|---|---|
| Number | PAM | gRNA | strand |
| 1 | AGG | TGATCCAGAACCCGGGCCCC | + |
| 2 | AGG | GAGCGTGCGCGAAGCGATCC | + |

TABLE 2
Potential high efficacy designed
sgRNAs for Androgen Receptor

| | | | gRNA |
|---|---|---|---|
| Number | PAM | gRNA | strand |
| 3 | AGG | CGATCCAGAACCCGGGCCCC | + |
| 4 | GGG | GGCGGCGGCGGCGGCGAGGC | + |
| 5 | GGG | GGCGGCGGCGGCGGCGAGGC | + |
| 6 | GGG | GGCGGCGGCGGCGGCGAGGC | + |
| 7 | GGG | GGCGGCGGCGGCGGCGAGGC | + |
| 8 | GGG | GGCGGCGGCGGCGGCGAGGC | + |
| 9 | GGG | GGCGGCGGCGGCGGCGAGGC | + |
| 10 | CGG | CGGCGGCGGCGGCGGCGAGG | + |
| 11 | CGG | CGGCGGCGGCGGCGGCGAGG | + |
| 12 | CGG | CGGCGGCGGCGGCGGCGAGG | + |
| 13 | CGG | CGGCGGCGGCGGCGGCGAGG | + |
| 14 | CGG | CGGCGGCGGCGGCGGCGAGG | + |
| 15 | CGG | CGGCGGCGGCGGCGGCGAGG | + |
| 16 | TGG | AAGTGGGCCAAGGCCTTGCC | + |
| 17 | TGG | AAGTGGGCCAAGGCCTTGCC | + |
| 18 | TGG | AAGTGGGCCAAGGCCTTGCC | + |
| 19 | TGG | TCTCCAGCTTGATGCGAGCG | − |
| 20 | TGG | TCTCCAGCTTGATGCGAGCG | − |
| 21 | TGG | TCTCCAGCTTGATGCGAGCG | − |
| 22 | TGG | TCTCCAGCTTGATGCGAGCG | − |
| 23 | TGG | TCTCCAGCTTGATGCGAGCG | − |
| 24 | TGG | TCTCCAGCTTGATGCGAGCG | − |
| 25 | TGG | ACACACTACACCTGGCTCAA | − |
| 26 | TGG | ACACACTACACCTGGCTCAA | − |
| 27 | TGG | ACACACTACACCTGGCTCAA | − |
| 28 | CGG | TAGCCCCCTACGGCTACACT | + |
| 29 | CGG | TAGCCCCCTACGGCTACACT | + |
| 30 | CGG | TAGCCCCCTACGGCTACACT | + |
| 31 | CGG | TAGCCCCCTACGGCTACACT | + |
| 32 | CGG | TAGCCCCCTACGGCTACACT | + |
| 33 | CGG | TAGCCCCCTACGGCTACACT | + |
| 34 | TGG | GCTCGCGGCCTCTGGGTGCC | − |
| 35 | TGG | GCTCGCGGCCTCTGGGTGCC | − |
| 36 | TGG | GCTCGCGGCCTCTGGGTGCC | − |
| 37 | TGG | GCTCGCGGCCTCTGGGTGCC | − |
| 38 | TGG | GCTCGCGGCCTCTGGGTGCC | − |
| 39 | AGG | CAGCAGCGGGAGAGCGAGGG | + |
| 40 | AGG | CAGCAGCGGGAGAGCGAGGG | + |
| 41 | AGG | CAGCAGCGGGAGAGCGAGGG | + |
| 42 | AGG | CAGCAGCGGGAGAGCGAGGG | + |
| 43 | AGG | CAGCAGCGGGAGAGCGAGGG | + |
| 44 | AGG | CAGCAGCGGGAGAGCGAGGG | + |
| 45 | GGG | TCTGGGACGCAACCTCTCTC | − |
| 46 | GGG | TCTGGGACGCAACCTCTCTC | − |
| 47 | GGG | TCTGGGACGCAACCTCTCTC | − |
| 48 | GGG | TCTGGGACGCAACCTCTCTC | − |
| 49 | GGG | TCTGGGACGCAACCTCTCTC | − |
| 50 | TGG | CTGCTGCGGCAGCCCCTTGC | − |
| 51 | TGG | CTGCTGCGGCAGCCCCTTGC | − |
| 52 | TGG | CTGCTGCGGCAGCCCCTTGC | − |
| 53 | TGG | CTGCTGCGGCAGCCCCTTGC | − |
| 54 | TGG | CTGCTGCGGCAGCCCCTTGC | − |
| 55 | AGG | GCCATCCAAACTCTTGAGAG | − |
| 56 | AGG | GCCATCCAAACTCTTGAGAG | − |
| 57 | AGG | GTCCTGGAAGCCATTGAGCC | + |
| 58 | AGG | GTCCTGGAAGCCATTGAGCC | + |
| 59 | AGG | GTCCTGGAAGCCATTGAGCC | + |
| 60 | TGG | CTGCTGCAGCAGCAGCAAAC | − |
| 61 | TGG | CTGCTGCAGCAGCAGCAAAC | − |
| 62 | TGG | CTGCTGCAGCAGCAGCAAAC | − |
| 63 | TGG | CTGCTGCAGCAGCAGCAAAC | − |
| 64 | TGG | CTGCTGCAGCAGCAGCAAAC | − |
| 65 | GGG | CGGCCCCCTCAGGGGCTGGC | + |
| 66 | GGG | CGGCCCCCTCAGGGGCTGGC | + |
| 67 | GGG | CGGCCCCCTCAGGGGCTGGC | + |
| 68 | GGG | CGGCCCCCTCAGGGGCTGGC | + |
| 69 | GGG | CGGCCCCCTCAGGGGCTGGC | + |
| 70 | GGG | CGGCCCCCTCAGGGGCTGGC | + |
| 71 | AGG | CTGGAGTGCCACCCCGAGAG | + |
| 72 | AGG | CTGGAGTGCCACCCCGAGAG | + |
| 73 | AGG | CTGGAGTGCCACCCCGAGAG | + |
| 74 | AGG | CTGGAGTGCCACCCCGAGAG | + |
| 75 | AGG | CTGGAGTGCCACCCCGAGAG | + |
| 76 | AGG | TCCGGAGTAGCTATCCATCC | − |
| 77 | AGG | TCCGGAGTAGCTATCCATCC | − |
| 78 | AGG | TCCGGAGTAGCTATCCATCC | − |
| 79 | AGG | TCCGGAGTAGCTATCCATCC | − |
| 80 | AGG | TCCGGAGTAGCTATCCATCC | − |
| 81 | AGG | TCCGGAGTAGCTATCCATCC | − |
| 82 | AGG | GCAGCAGCAGCGGGAGAGCG | + |
| 83 | AGG | GCAGCAGCAGCGGGAGAGCG | + |
| 84 | AGG | GCAGCAGCAGCGGGAGAGCG | + |
| 85 | AGG | GCAGCAGCAGCGGGAGAGCG | + |
| 86 | AGG | GCAGCAGCAGCGGGAGAGCG | + |
| 87 | AGG | GCAGCAGCAGCGGGAGAGCG | + |
| 88 | CGG | CGGCGGAGGGGGCGGCGGTC | − |
| 89 | CGG | CGGCGGAGGGGGCGGCGGTC | − |
| 90 | CGG | CGGCGGAGGGGGCGGCGGTC | − |
| 91 | CGG | CGGCGGAGGGGGCGGCGGTC | − |
| 92 | CGG | CGGCGGAGGGGGCGGCGGTC | − |
| 93 | CGG | CGGCGGAGGGGGCGGCGGTC | − |
| 94 | CGG | TCTCCCGCTGCTGCTGCCTT | − |
| 95 | CGG | TCTCCCGCTGCTGCTGCCTT | − |
| 96 | CGG | TCTCCCGCTGCTGCTGCCTT | − |
| 97 | CGG | TCTCCCGCTGCTGCTGCCTT | − |
| 98 | CGG | TCTCCCGCTGCTGCTGCCTT | − |
| 99 | CGG | TCTCCCGCTGCTGCTGCCTT | − |
| 100 | TGG | GGGGACCTGGCGAGCCTGCA | + |
| 101 | TGG | GGGGACCTGGCGAGCCTGCA | + |
| 102 | TGG | GGGGACCTGGCGAGCCTGCA | + |
| 103 | TGG | GGGGACCTGGCGAGCCTGCA | + |
| 104 | TGG | GGGGACCTGGCGAGCCTGCA | + |
| 105 | TGG | GGGGACCTGGCGAGCCTGCA | + |
| 106 | AGG | CCCGGGCCCCAGGCACCCAG | + |
| 107 | AGG | CCCGGGCCCCAGGCACCCAG | + |
| 108 | AGG | CCCGGGCCCCAGGCACCCAG | + |
| 109 | AGG | CCCGGGCCCCAGGCACCCAG | + |
| 110 | AGG | CCCGGGCCCCAGGCACCCAG | + |
| 111 | TGG | TTCCAGGACATTCAGAAAGA | − |
| 112 | TGG | TTCCAGGACATTCAGAAAGA | − |
| 113 | TGG | TTCCAGGACATTCAGAAAGA | − |
| 114 | CGG | CTCTGGGACGCAACCTCTCT | − |
| 115 | CGG | CTCTGGGACGCAACCTCTCT | − |
| 116 | CGG | CTCTGGGACGCAACCTCTCT | − |
| 117 | CGG | CTCTGGGACGCAACCTCTCT | − |
| 118 | CGG | CTCTGGGACGCAACCTCTCT | − |
| 119 | AGG | CTCCGGACTTGTAGAGAGAC | − |
| 120 | AGG | CTCCGGACTTGTAGAGAGAC | − |
| 121 | AGG | CTCCGGACTTGTAGAGAGAC | − |
| 122 | AGG | CTCCGGACTTGTAGAGAGAC | − |
| 123 | AGG | CTCCGGACTTGTAGAGAGAC | − |
| 124 | AGG | CTCCGGACTTGTAGAGAGAC | − |
| 125 | GGG | CCGCCGTGGCCGCCAGCAAG | + |
| 126 | GGG | CCGCCGTGGCCGCCAGCAAG | + |
| 127 | GGG | CCGCCGTGGCCGCCAGCAAG | + |
| 128 | GGG | CCGCCGTGGCCGCCAGCAAG | + |
| 129 | GGG | CCGCCGTGGCCGCCAGCAAG | + |
| 130 | GGG | GCTTGTACACGTGGTCAAGT | + |
| 131 | GGG | GCTTGTACACGTGGTCAAGT | + |
| 132 | GGG | GCTTGTACACGTGGTCAAGT | + |
| 133 | TGG | TAGAGGCCCCACAGGCTACC | + |
| 134 | TGG | TAGAGGCCCCACAGGCTACC | + |
| 135 | TGG | TAGAGGCCCCACAGGCTACC | + |
| 136 | TGG | TAGAGGCCCCACAGGCTACC | + |
| 137 | TGG | TAGAGGCCCCACAGGCTACC | + |
| 138 | CGG | GATGCTTGCAATTGCCAACC | − |
| 139 | CGG | GGACTACGGCAGCGCCTGGG | + |
| 140 | CGG | GGACTACGGCAGCGCCTGGG | + |
| 141 | CGG | GGACTACGGCAGCGCCTGGG | + |
| 142 | CGG | GGACTACGGCAGCGCCTGGG | + |
| 143 | CGG | GGACTACGGCAGCGCCTGGG | + |
| 144 | CGG | GGACTACGGCAGCGCCTGGG | + |
| 145 | GGG | TCCAAGGACAATTACTTAGG | + |
| 146 | GGG | TCCAAGGACAATTACTTAGG | + |
| 147 | GGG | TCCAAGGACAATTACTTAGG | + |
| 148 | GGG | TCCAAGGACAATTACTTAGG | + |
| 149 | GGG | TCCAAGGACAATTACTTAGG | + |
| 150 | GGG | TCCAAGGACAATTACTTAGG | + |
| 151 | TGG | AGCTTGTACACGTGGTCAAG | + |
| 152 | TGG | AGCTTGTACACGTGGTCAAG | + |
| 153 | TGG | AGCTTGTACACGTGGTCAAG | + |
| 154 | TGG | TAACCCCAAGCCATACTGCA | + |
| 155 | TGG | TTGACTTCTAGCAAATAAAT | − |
| 156 | TGG | GGTGAAGGATCGCCAGCCCA | − |
| 157 | TGG | GGTGAAGGATCGCCAGCCCA | − |
| 158 | AGG | CCGCTGCACCCGCGCCATGC | − |
| 159 | AGG | CCGCTGCACCCGCGCCATGC | − |
| 160 | AGG | CCGCTGCACCCGCGCCATGC | − |
| 161 | AGG | CCGCTGCACCCGCGCCATGC | − |
| 162 | AGG | CCGCTGCACCCGCGCCATGC | − |
| 163 | AGG | CCGCTGCACCCGCGCCATGC | − |
| 164 | AGG | GAAGCAGGGATGACTCTGGG | + |
| 165 | AGG | GAAGCAGGGATGACTCTGGG | + |
| 166 | AGG | GAAGCAGGGATGACTCTGGG | + |
| 167 | AGG | GAAGCAGGGATGACTCTGGG | + |
| 168 | TGG | CGCTGGACTACGGCAGCGCC | + |
| 169 | TGG | CGCTGGACTACGGCAGCGCC | + |
| 170 | TGG | CGCTGGACTACGGCAGCGCC | + |
| 171 | TGG | CGCTGGACTACGGCAGCGCC | + |
| 172 | TGG | CGCTGGACTACGGCAGCGCC | + |
| 173 | TGG | CGCTGGACTACGGCAGCGCC | + |
| 174 | GGG | CTCATTGAAAACCAGATCAG | − |
| 175 | GGG | CTCATTGAAAACCAGATCAG | − |
| 176 | TGG | TCCCCACGCTCGCATCAAGC | + |
| 177 | TGG | TCCCCACGCTCGCATCAAGC | + |
| 178 | TGG | TCCCCACGCTCGCATCAAGC | + |
| 179 | TGG | TCCCCACGCTCGCATCAAGC | + |
| 180 | TGG | TCCCCACGCTCGCATCAAGC | + |
| 181 | TGG | TCCCCACGCTCGCATCAAGC | + |
| 182 | GGG | ACAGGTACTTCTGTTTCCCT | − |
| 183 | GGG | ACAGGTACTTCTGTTTCCCT | − |
| 184 | GGG | ACAGGTACTTCTGTTTCCCT | − |
| 185 | GGG | ACAGGTACTTCTGTTTCCCT | − |
| 186 | GGG | ACAGGTACTTCTGTTTCCCT | − |
| 187 | GGG | TCCAGGACATTCAGAAAGAT | − |
| 188 | GGG | TCCAGGACATTCAGAAAGAT | − |
| 189 | GGG | TCCAGGACATTCAGAAAGAT | − |
| 190 | AGG | GGCTGTGAAGAGAGTGTGCC | − |
| 191 | AGG | GGCTGTGAAGAGAGTGTGCC | − |
| 192 | AGG | GGCTGTGAAGAGAGTGTGCC | − |
| 193 | AGG | GGCTGTGAAGAGAGTGTGCC | − |
| 194 | AGG | GGCTGTGAAGAGAGTGTGCC | − |
| 195 | AGG | GGCTGTGAAGAGAGTGTGCC | − |
| 196 | AGG | AGCCGCCGTGGCCGCCAGCA | + |
| 197 | AGG | AGCCGCCGTGGCCGCCAGCA | + |
| 198 | AGG | AGCCGCCGTGGCCGCCAGCA | + |
| 199 | AGG | AGCCGCCGTGGCCGCCAGCA | + |
| 200 | AGG | AGCCGCCGTGGCCGCCAGCA | + |
| 201 | TGG | TGTCATTCAGTACTCCTGGA | + |
| 202 | TGG | TGTCATTCAGTACTCCTGGA | + |

### Example 4

### LNP Characterizations and Morphology Analysis

The size, polydispersity index (PDI), and zeta potentials of AA3-DLin LNPs were measured in DI water using a Zeta Sizer dynamic light-scattering detector (15-mW laser, incident beam of 676 nm; Malvern, UK) at 25° C. and at a scattering angle of 900. The intensity-weighted mean value was recorded as the average of three measurements.

### Example 5

### Evaluation of LNP Transfection Efficacy by Fluorescence Microscopy and Sanger Sequencing

The in vitro transfection efficacy was evaluated in the HEK 293 cell line by delivering plasmid PX458 integrated with GFP, Cas9 and gRNA sequence, utilizing nanoparticles. Briefly, 1×10⁵ HEK 293 cells were seeded into each well of 24-well plate with 0.5 mL complete culture medium and incubated at 37° C. overnight to allow cell attachment. Then the LNPs were seeded at 24 h. The transfected GFP positive cells were observed under All-in-One Fluorescence Microscope (BZ-X710, Keyence, Japan) at predetermined time points with brightfield, fluorescent and merged channels using 10×PanFluor lens (Nikon, Japan). After observation and imaging, the cells were trypsinized by 0.25% Trypsin-EDTA (Sigma-Aldrich), followed by the addition of 0.5 mL 1×PBS and centrifuged to decant supernatant to obtain cell pellets. The cell pellets were resuspended in 0.5 mL PBS for flow cytometric analysis using a BD LSR II flow cytometer (BD Biosciences, San Jose, CA) and the data were analyzed using FACSDiva software (BD Biosciences, San Jose, CA). Data were acquired using a 488 nm laser with a 610/20 BP filter for the detection of GFP positive cells under a voltage of 350 V. At least 10,000 events were collected for each measurement. The cell DNA was then extracted via proteinase K lysis buffer, at an incubation temperature of 56° C. for 6 h followed by 95° C. for 10 minutes. Genomic DNA was subject to PCR for the Androgen Receptor gene in a Thermocycler (Bio-Rad) to amplify the target sequence. After PCR, the PCR product was purified by PCR purification kit (Qiagen) and sent to Azenta Biosciences for Sanger sequencing. The sequence analysis was performed with ICE analysis software (Synthego).

### Example 6

### In Vivo NP Delivery

All procedures for animal experiments were approved by the Rutgers University IACUC and performed in accordance with the NIH guidelines for the care and use of experimental animals. All the animals were ordered from Jackson Labs. Male C57BL/6 mice Balb/c mice (6 to 8 wks) were used for in vivo Luciferase mRNA (mLuc) (TriLink Biotechnologies) encapsulated NP screening and formulation optimization. Briefly, mLuc LNPs were rubbed onto skin of depilated mice at a concentration of 0.5 mg/kg mRNA. After 6-8 h, mice were treated with 100 μL D-Luciferin potassium salt (Xenolight) solution (15 mg/mL in PBS), anesthetized under isoflurane anesthesia, and measured by IVIS imaging system (Xenogen). For the hair regrowth experiment, mice were depilated with hair removal cream (Nair) and DHT at a concentration of 10⁻⁸M was applied to the skin daily, which slowed the regrowth of hair, to simulate DHT-mediated hair loss. The NPL-gRNA/cas9 treatment at a dosage of different ratios (e.g., at 5 ug sgRNA, 10 ug sgRNA, and 20 ug sgRNA, etc.) (gRNA:Cas9) was applied once immediately after depilation of hair, to the area of the back where hair was removed.

### Example 7

### Immunostaining and Histology

Tissue samples were fixed in 4% paraformaldehyde and dehydrated in 30% sucrose for 24 h and 48 h prior to embedding in optimal cutting temperature (OCT) compound, and immediately frozen at −80° C. The frozen OCT sample was sectioned into slices (8 m) using a cryotome and positioned onto glass slides. The tissue sections were incubated in 5% bovine serum albumin (BSA) blocking buffer at room temperature (r.t.) for 1 h and then washed with PBS. An androgen specific primary antibody (1:200 diluted in 2% BSA buffer, Abcam) was applied and incubated at 4° C. overnight. The slices were rinsed with PBS for three times at 10 minutes each and then stained with 488 nm Goat anti-rabbit secondary antibody (1:500, Abcam) and incubated at room temperature for 2 hours followed by rinsing with PBS for three times at 10 minutes each. For histology, the tissue sections were incubated in Hematoxylin, Mayer's (Lillie's Modification) (abcam) to completely cover the tissue section for 5 mins. Slides were rinsed in two changes of distilled water. Bluing reagent (abcam) was added to the slides for 15 seconds. Slides were rinsed in two changes of distilled water. Pure alcohol was used to rinse the slides. Then Eosin Y Solution (Modified Alcoholic) (abcam) was added to slides and incubated for 3 minutes. Slides were then rinsed in three changes of pure alcohol.

### Example 8

### Results

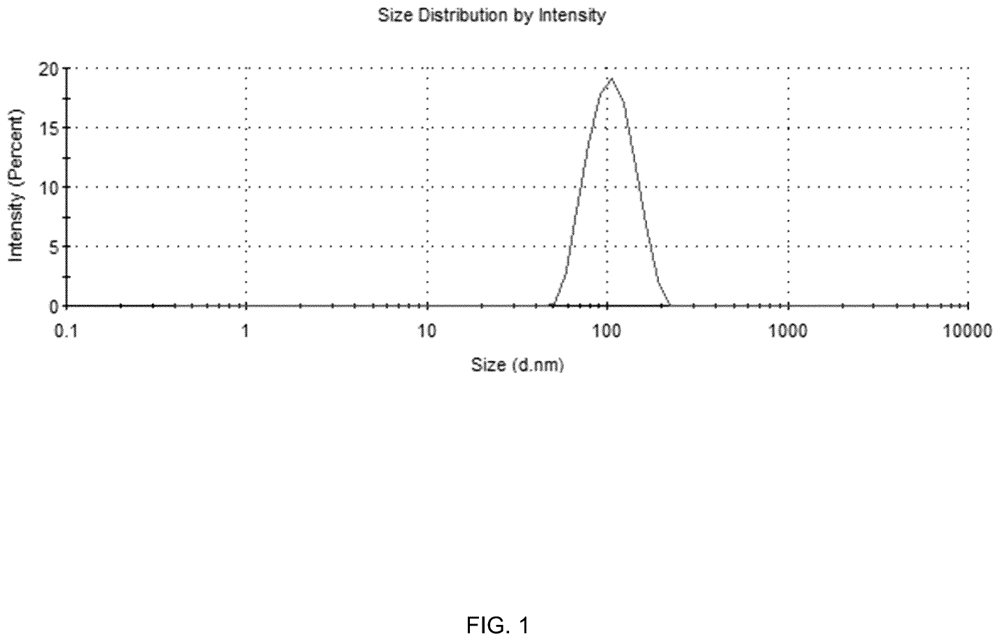

FIG. 1 depicts the size and distribution of LNPs containing sgRNA/Cas9 mRNA. The successful creation of nanoparticles approximately 100 nm in size is shown. Nanoparticles under 200 nm are more effective at entering cells for transfection purposes, which is beneficial for the gene editing efficacy. This means that the created nanoparticles of the present disclosure have a higher likelihood to enter cells and deliver the CRISPR-Cas/gRNA payload of the present disclosure.

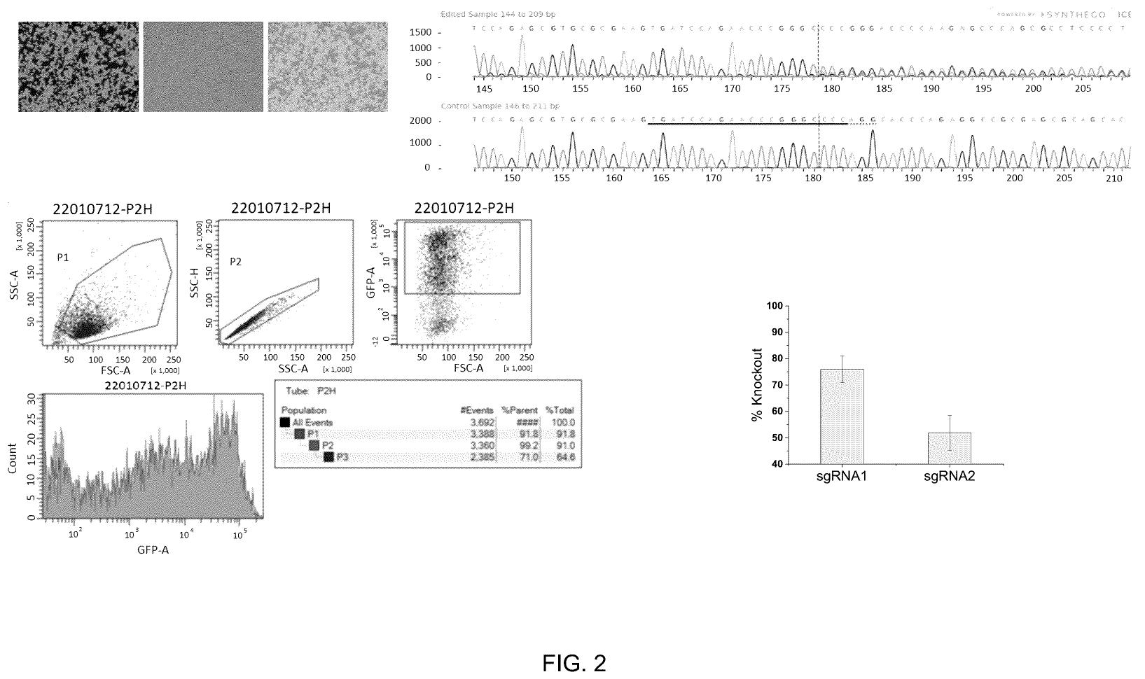

FIG. 2 shows the efficacy of transfection in HEK 293T cells as well as the knockout percentage of the plasmid containing GFP, Cas9, and gRNA. Over 70% of cells were successfully transfected with gRNA/Cas9 complex, with almost 90% having at least a weak GFP signal. GFP-positive cells were sorted out and analyzed for knockout. The DNA trace shown indicates the region where edits were designed to target. The dashed line indicates the protospacer adjacent motif (PAM), a DNA sequence immediately following the DNA sequence targeted by the Cas9 nuclease. To the left of the PAM, the sequence appears as one coherent sequence, and to the right of the PAM, it is clear that there are multiple sequences, indicated by the shorter and more numerous peaks. This indicates that the sanger sequencing detects multiple divergent sequences after the PAM site, indicating that insertions or deletions have been made, which indicate potential knockouts. These knockouts were analyzed by Inference of CRISPR Edits (ICE) tool, which indicates that the top performing sgRNAs, sgRNA1 (P6) (e.g., SEQ ID NO: 1) and sgRNA2 (P2) (e.g., SEQ ID NO: 2) had around a 76% and 51% knockout, respectively. These results indicate successful in vitro delivery and editing of the present lipid nanoparticle system. (Top left) Sample image of transfection of HEK 293T cells at 48 h with plasmids containing GFP reporter gene as well as sgRNA and CRISPR/Cas9. (Bottom left) Sample FACS cell sorting data indicating percentage of GFP positive cells, which were utilized for sequencing analysis. (Top right) Sample DNA sequence trace of sgRNAs. Vertical dashed line represents the predicted CRISPR edit site. (Bottom right) Knockout percentages of sgRNAs, calculated by ICE analysis (n=5). This indicates the percentage of transfected cells which show a knockout mutation (insertion or deletion).

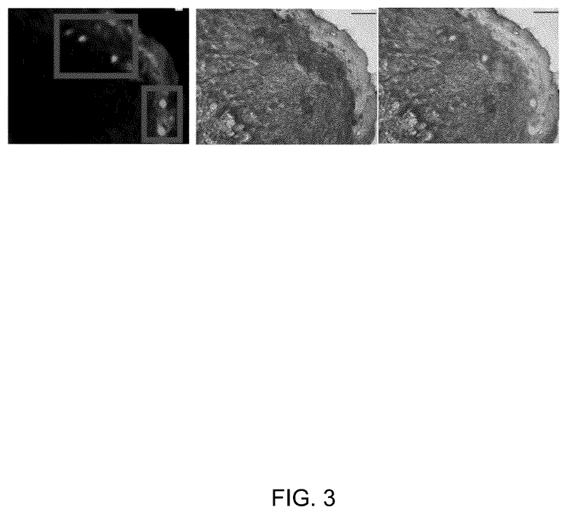

FIG. 3 shows the skin layer of mice treated topically with DSPC-FITC labeled LNPs after depilation. DSPC-FITC labeled LNPs permeate the skin layer after depilation. The results show that LNPs accumulate in the hair follicles and general penetration of the skin is up to 150 microns in depth. This indicates that LNPs are able to accumulate in the sites of interest for amelioration of baldness through dissolved hair follicles. LNPs were administered topically on the skin of mice after depilation and images captured 24 h after treatment, indicating penetration depth of LNPs are 100-150 microns. Scale bar represents 100 microns.

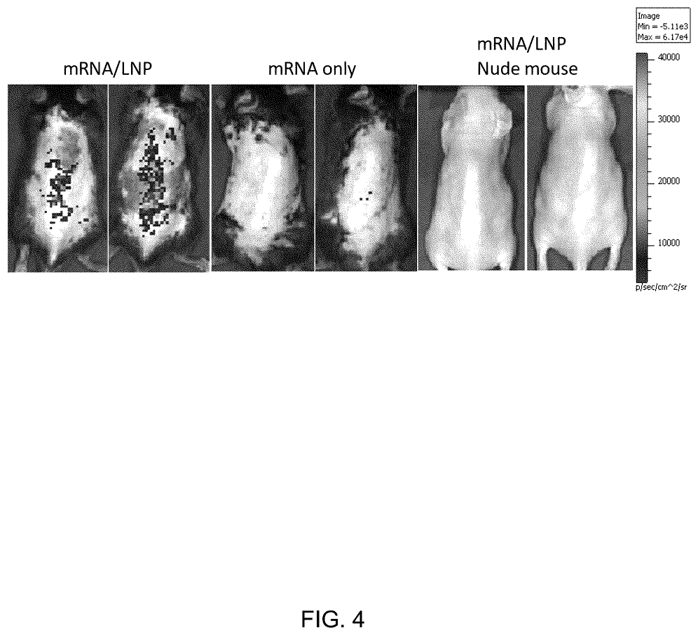

FIG. 4 shows that the LNPs, according to some embodiments of the present disclosure, are able to deliver luciferase mRNA transdermally when rubbed onto the skin after depilation. This means that the delivered mRNAs are not only accumulating in the skin, but they are actively being taken up by the cells and translated into protein, which can be detected by the IVIS imaging system. Delivery is presumably through the pores generated by depilation cream in C57BL/6 mice, which dissolves hair follicles which previously occupied the area. LNPs loaded with luciferase mRNA (left), empty (no mRNA) LNPS (middle) and nude mice (no hair removal).

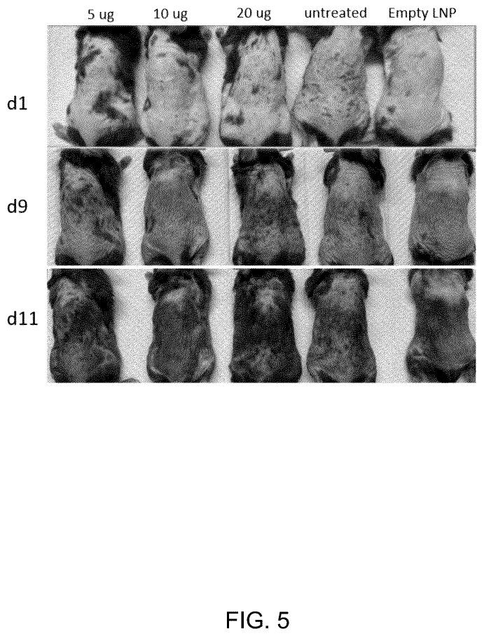

FIG. 5 shows a study of LNP/CRISPR dose dependent hair regrowth in a mouse model of baldness, according to some embodiments of the present disclosure. The data suggests that a higher dosage of sgRNA/Cas9 LNPs increased the hair regrowth of mice, which were treated with DHT to inhibit the hair growth. The aim of the LNP delivery system is to disrupt the DHT binding to the androgen receptor through knockout of this receptor, which would render DHT ineffective in the treated regions. C57ZBL mice (age 6-8 weeks) were depilated on the back and treated with LNPs containing sgRNA and CRISPR/Cas9 mRNA at 1:1 molar ratio, one time. On day 1 until the end of the experiment, all mice were treated with DHT (10-8 M) daily (200 uL, topical) to inhibit hair formation. From left to right, 5 ug sgRNA, 10 ug sgRNA, 20 ug sgRNA, no LNP, and empty LNP.

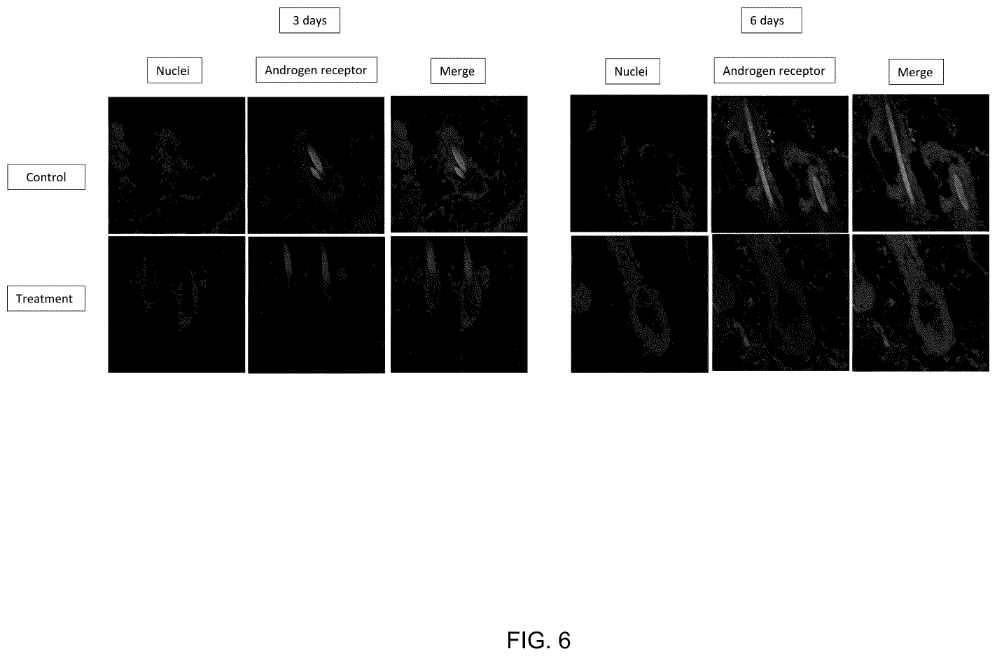

FIG. 6 depicts an immunostaining image of androgen receptor expression in mice 3 days (left) and 6 days (right) after treatment and after hair removal, according to some embodiments of the present disclosure. The images show that the androgen receptor (green) is localized in the follicles of the control mice, and that conversely in sgRNA/Cas9 LNP treated mice, the androgen receptor shows lower expression in the follicles. This indicates that the follicle cells are taking up the gene editing sgRNA/Cas9 LNPs and that the androgen receptor is being knocked down in these cells. Immunostaining image. (Top row) control untreated mice and (Bottom row) 20 ug sgRNA targeting androgen receptor.

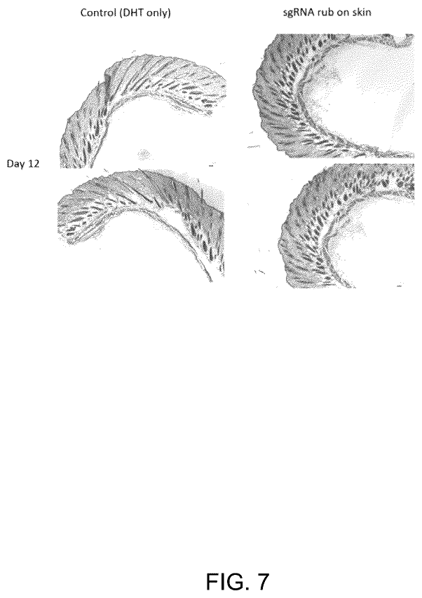

FIG. 7 shows histology of mouse skin and hair according to some embodiments of the present disclosure. There is a higher number and density of hairs in mouse skin after 12 days, in the sgRNA/Cas9LNP treated mice over the control mice. (Left column) control untreated. (Right column) treated with 20 ug sgRNA, 20 ug Cas9 encapsulated in LNP.

The Examples above demonstrate that the CRISPR-Cas9/sgRNAs according to one or more embodiments of the present disclosure knocks out the androgen receptor via editing of DNA sequence in vitro. The LNP delivery system according to one or more embodiments of the present disclosure permeates the skin layer and accumulate in target hair follicles, and is able to deliver mRNA through the skin. In addition, the LNPs carrying CRISPR-Cas9/sgRNA show dose dependent delivery in a preliminary mouse model of baldness. The treatment reduced the expression of androgen receptor in follicles of mice. The treatment also increased the density of hairs in treated mice. Collectively, this data suggests a successful and promising treatment for baldness mediated by CRISPR-Cas/LNP delivery system according to one or more embodiments of the present disclosure.

### REFERENCES

ADDIN EN.REFLIST Birch, M. P., et al. (2001). “Hair density, hair diameter and the prevalence of female pattern hair loss.” Br J Dermatol 144(2): 297-304.
Fabbrocini, G., et al. (2018). “Female pattern hair loss: A clinical, pathophysiologic, and therapeutic review.” Int J Womens Dermatol 4(4): 203-211.
Lolli, F., et al. (2017). “Androgenetic alopecia: a review.” Endocrine 57(1): 9-17.
Messenger, A. (2008). Male Androgenetic Alopecia. Hair Growth and Disorders: 159-170.
Rathnayake, D. and R. Sinclair (2010). “Male androgenetic alopecia.” Expert Opin Pharmacother 11(8): 1295-1304.
York, K., et al. (2020). “A review of the treatment of male pattern hair loss.” Expert Opinion on Pharmacotherapy 21(5): 603-612.

What is claimed is:

1. A nucleic acid construct comprising a domain hybridizing with early coding regions of the androgen receptor gene.

2. The nucleic acid construct of claim 1, wherein the domain hybridizes with the first coding region of the androgen receptor gene.

3. The nucleic acid construct of claim 2, wherein the domain hybridizes with the first coding exon of the androgen receptor gene.

4. The nucleic acid construct of claim 1, wherein the domain comprises any one sequence selected from the group consisting of SEQ ID NOs: 1-202.

5. A polynucleotide encoding the nucleic acid construct of claim 1 and a CRISPR-Cas nuclease, wherein the nucleic acid construct and the CRISPR-Cas nuclease form a complex that hybridizes with the androgen receptor gene.

6. The polynucleotide of claim 5, wherein the polynucleotide is codon optimized for expression in a mammal.

7. The polynucleotide of claim 6, wherein the CRISPR-Cas is CRISPR-Cas9.

8. An expression cassette or vector comprising the polynucleotide of claim 5.

9. A cell comprising at least one of the nucleic acid construct of claim 1, an expression cassette or vector comprising the same, a polynucleotide encoding the nucleic acid construct of claim 1 and a CRISPR-Cas nuclease, and an expression cassette or vector comprising the same.

10. The cell of claim 9, wherein the cell is a mammalian cell.

11. A method of treating baldness or hair loss in a subject in need thereof, the method comprising administering to the subject a therapeutically effective amount of:
(a) the nucleic acid construct of claim 1 and/or an expression cassette or vector comprising the same; and
(b) a CRSIPR/Cas nuclease and/or an expression cassette or vector comprising the same;
wherein the nucleic acid construct forms a complex with the CRISPR/Cas nuclease, the complex hybridizing with early coding regions of the androgen receptor gene, wherein the complex knocks out the gene.

12. The method of claim 11, wherein the method is a one-time administration.

13. The method of claim 11, wherein the method comprises multiple administrations.

14. The method of claim 11, wherein the administering is transdermal, topical, or subcutaneous.

15. A composition comprising:
(a) a nanoparticle delivery system; and
(b) at least one of the nucleic acid construct of claim 1, an expression cassette or vector comprising the same, a polynucleotide encoding the nucleic acid construct of claim 1 and a CRISPR-Cas nuclease, and an expression cassette or vector comprising the same.

16. The composition of claim 15, wherein the nanoparticle delivery system comprises lipid nanoparticles (LNP) and an ionizable cationic lipid compound, wherein the ionizable cationic lipid compound is synthesized by reacting an amino alcohol, at least one fatty acid, and at least one lipid component.

17. The composition of claim 16, wherein the amino acid alcohol is piperazine or a derivative thereof.

18. The composition of claim 16, wherein the at least one fatty acid is a C4-C26 fatty acid.

19. The composition of claim 18, wherein the at least one fatty acid is selected from the group consisting of octanoic acid (C8), decanoic acid (C10), dodecanoic acid (C12), tetradecanoic acid (C14), hexadecanoic acid (C16), octadecanoic acid (C18), oleic acid (C18:1), linoleic acid (C18:2), and a combinations thereof.

20. The composition of claim 16, wherein the at least lipid component is selected from the group consisting of helper phospholipids, PEGylated lipids, cholesterol, and a combination thereof.
